(19) United States
(12) Patent Application Publication
(10) Pub. No.: US 2017/0231659 A1
(43) Pub. Date: Aug. 17, 2017

(54) EMBRYO TRANSFER CATHETERS

(71) Applicant: The Cooper Companies Global Holdings LP, St. Michael (BB)

(72) Inventors: Stephen James Field, Kent (GB); Timothy Bateman, London (GB); Andrew Thomas Jeffrey, Kent (GB); Christopher John Woosnam, Cheshire (GB)

(21) Appl. No.: 15/430,749

(22) Filed: Feb. 13, 2017

(30) Foreign Application Priority Data
Feb. 13, 2016 (GB) 1602689.0

(51) Int. Cl. A61B 17/435; A61M 25/00

(52) U.S. Cl. CPC A61B 17/435; A61M 25/0043; A61M 25/0097; A61M 2025/0018

(57) ABSTRACT

A transfer device includes an elongate tube defining a lumen configured to support an organism and an obstruction positioned along the elongate tube. The obstruction is configured to prevent the organism from moving proximally past the obstruction, such that the obstruction defines a distal end region of the elongate tube in which the organism can move along the lumen of the elongate tube.

### CROSS-REFERENCE TO RELATED APPLICATION

This application claims priority under 35 U.S.C. §119 to Great Britain Patent Application 1602689.0, filed on Feb. 13, 2016, the content of which is incorporated herein by reference in its entirety.

### TECHNICAL FIELD

The present disclosure relates to transfer devices, and more particularly to embryo transfer catheters.

### BACKGROUND

Embryo transfer catheters are used during in vitro fertilization (IVF) procedures to transfer an embryo from a culture dish in an incubator to a patient. An embryo is drawn into a catheter by suction applied by a syringe connected to a proximal end (e.g., a machine end) of the catheter. One problem that can occur is that the embryo may migrate along the catheter towards the proximal end. Such migration increases the risk that an embryo transfer will fail (i.e., the embryo may remain in the catheter instead of being expelled into the uterus of the patient). In order to reduce this risk, the catheter and syringe may be held horizontal. Alternatively, the embryo and a small amount of culture fluid in which the embryo is suspended may be held between two air bubbles within the catheter. Neither of these techniques completely removes the risk of the embryo migrating towards the proximal end of the catheter.

### SUMMARY

The present disclosure provides an embryo transfer catheter including a means positioned along the catheter to restrict passage of an embryo.

The means to restrict passage may include a plug that allows passage of liquid but prevents passage of an embryo. The plug may be formed from a gauze, a foam, or sintered plastics. The plug may be coated with a non-stick coating to reduce the risk that an embryo will stick to the plug. Alternatively, the means to restrict passage may include a region along the catheter having a reduced internal diameter sufficient to reduce the ability of an embryo to pass. In a further alternative embodiment, the means to restrict passage may include a region along the catheter with a tortuous passage effective to reduce migration of the embryo along the region. The tortuous passage may be provided by the region having a twisted, helical formation.

In one aspect of the invention, a transfer device includes an elongate tube defining a lumen configured to support an organism and an obstruction positioned along the elongate tube. The obstruction is configured to prevent the organism from moving proximally past the obstruction, such that the obstruction defines a distal end region of the elongate tube in which the organism can move along the lumen of the elongate tube.

In some embodiments, the organism is an embryo.

In certain embodiments, the lumen of the elongate tube is configured to support the organism suspended in a liquid medium.

In some embodiments, the obstruction defines one or more openings sized to allow passage of the liquid medium and sized to prevent passage of the organism.

In certain embodiments, the obstruction is a plug located within the lumen of the elongate tube.

In some embodiments, the plug includes holes that are sized to allow passage of a liquid medium suspending the organism and that are sized to prevent passage of the organism.

In certain embodiments, the plug is made of one or more materials including a fine gauze, a foam, and a sintered plastic.

In some embodiments, the plug includes a non-stick coating.

In certain embodiments, the obstruction is a restriction positioned along a wall of the elongate tube.

In some embodiments, an internal diameter of the wall at the restriction is less than an internal diameter of the wall at a location distal to the restriction.

In certain embodiments, an outer diameter of the wall at the restriction is equal to an outer diameter of the wall at locations proximal and distal to the restriction.

In some embodiments, the restriction is formed from a sleeve or a liner positioned within the lumen of the elongate tube.

In certain embodiments, the restriction has a diameter that is less than about 0.05 mm.

In some embodiments, the obstruction is a tortuous path positioned along a wall of the elongate tube.

In certain embodiments, the tortuous path provides a helical path of the lumen of the elongate tube.

In some embodiments, the obstruction is located about 40 mm to about 60 mm from a distal end of the elongate tube.

In certain embodiments, the elongate tube is made of one or more materials including a thermoplastic polyurethane, a polyolefin plastomer, and a polyvinyl chloride (PVC) material with a non-toxic plasticizer.

In some embodiments, the elongate tube has a length of about 910 mm.

In certain embodiments, the elongate tube has a maximum external diameter of about 1.6 mm and a maximum internal diameter of about 1.1 mm.

In some embodiments, the transfer device further includes a hub located at a proximal end of the elongate tube and configured to connect to a suction apparatus.

### DESCRIPTION OF DRAWINGS

FIG. 1 is a side elevation view of an assembly of an embryo transfer catheter and a syringe.

FIG. 2 is an enlarged cross-sectional side elevation view of one form of the catheter of FIG. 1.

FIG. 3 is an enlarged cross-sectional side elevation view of an alternative form of the catheter of FIG. 1.

FIG. 4 is an enlarged side elevation view of a further alternative form of the catheter of FIG. 1.

Like reference symbols in the various figures indicate like elements. In some examples, illustrations shown in the drawings may not be drawn to scale.

### DETAILED DESCRIPTION

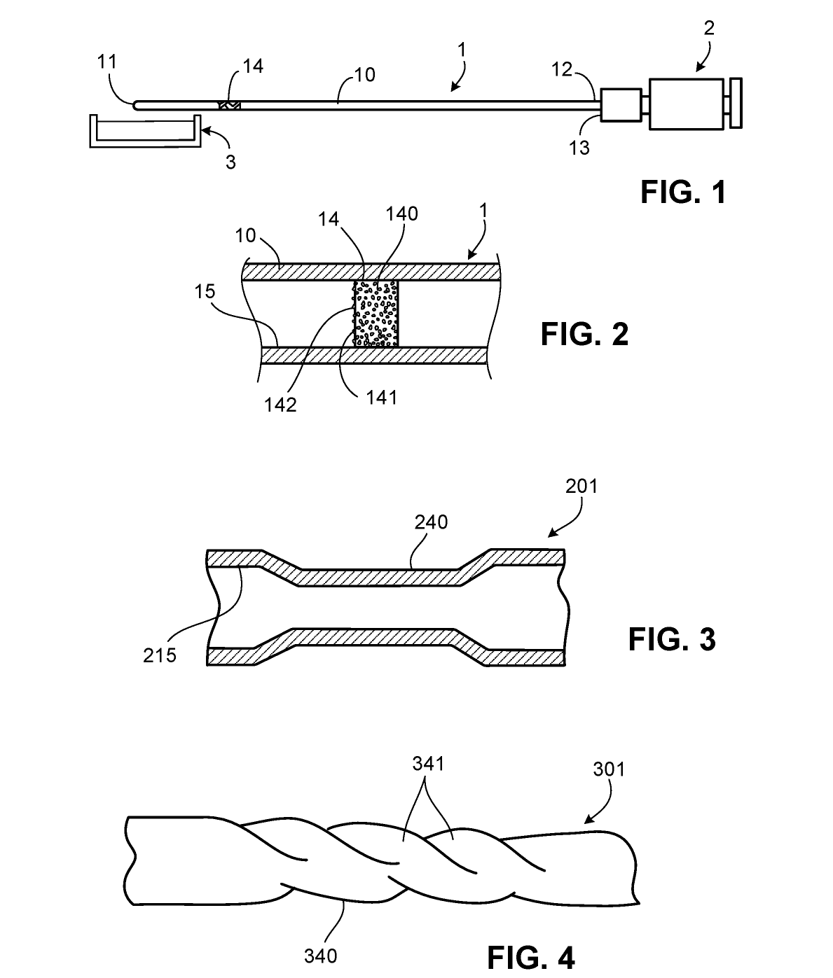

With reference first to FIGS. 1 and 2, there is shown an embryo transfer catheter 1, a syringe 2 connected to a proximal end 12 (e.g., a machine end) of the catheter, and a culture dish 3 containing a liquid culture medium and an embryo. The catheter 1 has a shaft 10 that is made of a flexible, biocompatible plastics material, such as thermoplastic polyurethane. In some embodiments, the shaft 10 may be made of other materials, such as a polyolefin plastomer or a polyvinyl chloride (PVC) material with a non-toxic plasticizer. The catheter 1 typically has a length of about 910 mm, has a circular cross section, has an external diameter of about 1.6 mm, and has an internal diameter of about 1.1 mm. In some embodiments, the catheter 1 may have a different cross-sectional shape. The shaft 10 has a distal end 11 (e.g., a patient end) that is open, smooth, and round. The proximal end 12 of the shaft 10 is attached to a hub 13 having a female coupling bore into which a nose of the syringe 2 is sealingly fitted.

The catheter 1 differs from conventional embryo transfer catheters in that the catheter 1 has some form of a means 14 at a location along the catheter 1 to prevent migration of an embryo or to substantially reduce the risk that the embryo will migrate past the location. This means 14 for restricting passage of an embryo is located about 40 mm to about 60 mm from the distal end 11 of the catheter 1. The means 14 can take various different forms.

FIG. 2 shows an arrangement where the means 14 for restricting passage is in the form of a plug 140 secured in a bore 15 of the shaft 10. The plug 140 is made of a fine gauze, a foam, a sintered plastics, or another type of material that allows a flow of liquid through the plug 140, but has holes that are sufficiently small to prevent an embryo from passing through the plug 140. At a transfer stage, embryos typically have a width of about 0.1 mm to about 0.2 mm. The plug 140 (e.g., at least a distal end surface 141 of the plug 140 exposed for contact with the embryo) may be coated with a layer 142 of non-stick material (e.g., polytetrafluoroethylene (PTFE) or another fluoropolymer) to minimize the risk of the embryo sticking to the plug 140. In some embodiments, the layer 142 may be made of other materials, such as expanded PTFE (ePTFE), which is both non-stick and porous, or sintered polyethylene or polypropylene. Accordingly, a configuration of the catheter 1 ensures that the embryo remains within a distal end portion of the catheter 1 that is between the patient end surface 141 of the plug 140 and the distal end 11 of the catheter 1. This configuration ensures that, when a plunger of the syringe 2 is depressed, the embryo is expelled from the distal end 11 of the catheter 1 into the uterus of the patient.

There are alternative arrangements by which migration of the embryo along the catheter can be prevented or reduced. For example, FIG. 3 shows a part of an alternative catheter 201 that has a short region 240 along which an internal diameter of the catheter 1 is reduced (e.g., to a diameter that is less than that of the embryo, such as between about 0.1 mm and about 0.05 mm). This reduced internal diameter is effective to prevent passage of the embryo along the region 240. FIG. 3 shows an arrangement where a bore 215 of the catheter 201 is reduced by necking an outer wall of the catheter 201 to reduce both an external diameter and the internal diameter of the catheter 201. Alternatively, the external diameter of the catheter 201 could be maintained along its length, while the internal diameter is reduced (e.g., by inserting a sleeve or a liner into the bore 215 of the catheter 201 along a region where the internal diameter needs to be reduced).

In another embodiment, migration of the embryo is prevented or reduced by a tortuous path that is provided along a catheter over a migration-reducing region. An example of such a tortuous path 341 is shown in FIG. 4, where a catheter 301 is twisted about its axis along a region 340 to form a helical path for a bore of the catheter 301 along the region 340. It is more difficult for an embryo to travel along such a helical path or otherwise tortuous path because the embryo would have to change a migration direction to navigate the path, which is a difficult endeavor for an embryo.

A number of embodiments have been described. Nevertheless, it will be understood that various modifications may be made without departing from the spirit and scope of the present disclosure. Accordingly, other embodiments are within the scope of the following claims.

What is claimed is:

1. A transfer device, comprising:
an elongate tube defining a lumen configured to support an organism; and
an obstruction positioned along the elongate tube and configured to prevent the organism from moving proximally past the obstruction, such that the obstruction defines a distal end region of the elongate tube in which the organism can move along the lumen of the elongate tube.

2. The transfer device of claim 1, wherein the organism is an embryo.

3. The transfer device of claim 1, wherein the lumen of the elongate tube is configured to support the organism suspended in a liquid medium.

4. The transfer device of claim 3, wherein the obstruction defines one or more openings sized to allow passage of the liquid medium and sized to prevent passage of the organism.

5. The transfer device of claim 1, wherein the obstruction comprises a plug located within the lumen of the elongate tube.

6. The transfer device of claim 5, wherein the plug includes holes that are sized to allow passage of a liquid medium suspending the organism and that are sized to prevent passage of the organism.

7. The transfer device of claim 5, wherein the plug is made of one or more materials including a fine gauze, a foam, and a sintered plastic.

8. The transfer device of claim 5, wherein the plug comprises a non-stick coating.

9. The transfer device of claim 1, wherein the obstruction comprises a restriction positioned along a wall of the elongate tube.

10. The transfer device of claim 9, wherein an internal diameter of the wall at the restriction is less than an internal diameter of the wall at a location distal to the restriction.

11. The transfer device of claim 10, wherein an outer diameter of the wall at the restriction is equal to an outer diameter of the wall at locations proximal and distal to the restriction.

12. The transfer device of claim 9, wherein the restriction is formed from a sleeve or a liner positioned within the lumen of the elongate tube.

13. The transfer device of claim 9, wherein the restriction has a diameter that is less than about 0.05 mm.

14. The transfer device of claim 1, wherein the obstruction comprises a tortuous path positioned along a wall of the elongate tube.

15. The transfer device of claim 1, wherein the tortuous path comprises a helical path of the lumen of the elongate tube.

16. The transfer device of claim 1, wherein the obstruction is located about 40 mm to about 60 mm from a distal end of the elongate tube.

17. The transfer device of claim 1, wherein the elongate tube is made of one or more materials including a thermoplastic polyurethane, a polyolefin plastomer, and a polyvinyl chloride (PVC) material with a non-toxic plasticizer.

18. The transfer device of claim 1, wherein the elongate tube has a length of about 910 mm.

19. The transfer device of claim 1, wherein the elongate tube has a maximum external diameter of about 1.6 mm and a maximum internal diameter of about 1.1 mm.

20. The transfer device of claim 1, further comprising a hub located at a proximal end of the elongate tube and configured to connect to a suction apparatus.
